(19) United States
(12) Patent Application Publication
(10) Pub. No.: US 2022/0373437 A1
(43) Pub. Date: Nov. 24, 2022

(54) SYSTEM FOR EVALUATING CHEMO-PREVENTIVE POTENTIAL OF PHC AND ITS PREPARED CHITOSAN NANOPARTICLES

(71) Applicants: Tarique Mahmood Ansari, Lucknow (IN); Saba Parveen, Lucknow (IN); Mohammad Haris Siddiqui, Lucknow (IN); Tanveer Wani, Riyadh (SA); Seema Zargar, Riyadh (SA); Farogh Ahsan, Lucknow (IN); Arshiya Shamim, Lucknow (IN); Mohammad Shariq, Lucknow (IN); Vaseem Ahmad Ansari, Lucknow (IN); Anuradha Mishra, Lucknow (IN); Alvina Farooqui, Lucknow (IN)

(72) Inventors: Tarique Mahmood Ansari, Lucknow (IN); Saba Parveen, Lucknow (IN); Mohammad Haris Siddiqui, Lucknow (IN); Tanveer Wani, Riyadh (SA); Seema Zargar, Riyadh (SA); Farogh Ahsan, Lucknow (IN); Arshiya Shamim, Lucknow (IN); Mohammad Shariq, Lucknow (IN); Vaseem Ahmad Ansari, Lucknow (IN); Anuradha Mishra, Lucknow (IN); Alvina Farooqui, Lucknow (IN)

(21) Appl. No.: 17/816,792

(22) Filed: Aug. 2, 2022

(51) Int. Cl. G01N 1/28; G01N 33/50; G01N 21/33

(52) U.S. Cl. CPC G01N 1/286; G01N 33/5011; G01N 21/33; G01N 2001/2866

(57) ABSTRACT

A system for treating cancer and evaluating chemo-preventive potential of PHC and its prepared chitosan nanoparticles is described. The rats are divided into eight groups, from which group 1 is served as normal control, and group 2-8 are given single dose of DEN and repeated dose of CCl₄, wherein freshly prepared solution of DEN in normal saline is used for the induction of HCC in rats by administering 200 mg/kg, i.p., PHC (2:1:1) in normal saline suspension to administer at doses of 900 mg/kg, wherein serum and tissue samples are collected after anesthetizing overnight fasted rats using intraperitoneal administration of thiopentone sodium at a dose of 40 mg/kg, wherein the collected serum and tissue samples is treated and thereby the chemo-preventive potential of PHC (2:1:1) and its prepared chitosan nanoparticles is evaluated upon determining liver markers, antioxidant parameters, total bilirubin, protein, lipid peroxidation, and liver cancer biomarkers.

### TECHNICAL FIELD

The present disclosure relates to a system to estimate the potential of selected polyherbal combination and its prepared chitosan nanoparticles against DEN induced liver tumors in rats, in more details, the system delivers new perceptions into the development of different formulations of PHC with a ratio of 2:1:1 which can act as targeted preventive therapies for hepatocellular carcinoma.

### BACKGROUND

Chemoprevention is well-defined as the use of synthetic or natural products that obstruct the growth and expansion of cancer cells likewise by blocking DNA damage or by arresting the development of pre-malignant cells. Hepatocellular carcinoma or HCC, reported to be the utmost common malignant neoplasms is most important reason of morbidity and mortality Globally.

Chemoprevention is the best way to control HCC, by which the prevalence of cancer could be prohibited or reversed considerably by administering single or combined naturally occurring and/or synthetic anti-carcinogens. The prospective of inhibiting tumor development in targeted, highly risky, diseased population as well as in general population has been increased considerably. There is an emergent requirement to develop a novel chemo-therapeutic/preventive approach to enhance patient survival owing to poor prediction and high reoccurrence of hepatocellular carcinoma. The studies determining the therapeutic role of natural products are of vast importance because of biomedical applications of the active principle engaged as well as to understand the processes of homeostatic system in living beings. Natural food comprises numerous ingredients that are capable of scavenging reactive oxygen species directly or indirectly by encouraging mechanisms, which increase detoxification. Natural products are of great interest due to their safety and fewer side-effects and have been considered for development as cancer chemotherapeutic agents.

Diethylnitrosamine or N-Nitrosodiethylamine (DEN/NDEA) is a strong and powerful genotoxic nitrosamine which is recognized to encourage impairment of the nuclear enzymes that are involved in DNA repair or replication and is characteristically used in experimental models to originate liver carcinoma. It has been confirmed that Diethylnitrosamine is metabolized into its active metabolite (ethyl radical) that cooperates with DNA instigating its mutation and chromosomal aberrations that would ultimately results in the growth and expansion of hepatocarcinogenesis. Furthermore, the active metabolites of DEN produced via cytochrome isoform 2E1 (CYP 2E1) escalate the level of oxidative stress that leads to cytotoxicity. These N-Nitroso complexes are reported to act as strong and powerful carcinogens in several primates. Foods such as meat and milk products, salted fish, alcoholic beverages and some vegetables are principal sources of nitroso compounds. Thus, numerous factors that exist in the diet can transform these levels by acting as a catalytic agent or inhibitors and play an essential role in carcinogenesis. In human beings, the regular intake of volatile nitrosamine from food is approximately 1 μg/day.

There exists a need to have a system for evaluating chemo-preventive potential of PHC and its prepared chitosan nanoparticles in an efficient manner.

### BRIEF SUMMARY

The present disclosure seeks to provide a system for treating cancer and evaluating chemo-preventive potential of PHC and its prepared chitosan nanoparticles.

In an embodiment, a system for treating cancer and evaluating chemo-preventive potential of PHC and its prepared chitosan nanoparticles is disclosed. The system includes a nanoparticle synthesis unit for synthesizing chitosan nanoparticles using polyherbal combination of ratio (2:1:1). The system further includes a selection unit for selecting a group of rats each weighing 125-150 grams and accommodating in polypropylene cages under proper environmental conditions at room temperature of 23±2° C. and 50-60% relative humidity followed by 12 hours light/dark cycle. The system further includes an injection for inducing Hepatocellular Carcinoma (HCC), wherein for the induction of HCC, rats administered single intraperitoneal injection of DEN (200 mg/kg body weight) followed by weekly subcutaneous injections of carbon tetrachloride (3 ml/kg body weight) for 6 weeks, wherein the rats are divided into eight groups, from which group 1 is served as normal control, and group 2-8 are given single dose of DEN and repeated dose of CCl₄ till the completion of the experimental duration of 16 weeks, wherein freshly prepared solution of DEN in normal saline is used for the induction of HCC in rats by administering 200 mg/kg, i.p., PHC (2:1:1) is dispersed in normal saline suspension to administer at doses of 900 mg/kg, p.o and 1935 mg/kg p.o. Soranib (Sorafenib 200 mg) tablets are powdered and dissolve in normal saline solution and the freshly prepared solution is used as standard drug. The system further includes a collection unit for collecting serum and tissue samples after anesthetizing overnight fasted rats using intraperitoneal administration of thiopentone sodium at a dose of 40 mg/kg, wherein the blood sample is collected by retro-orbital plexus followed by cardiac puncture. The system further includes a treatment unit for treating the collected serum and tissue samples. The system further includes an analyzer equipped with a spectrophotometer for evaluating the chemo-preventive potential of PHC (2:1:1) and its prepared chitosan nanoparticles upon determining liver markers, antioxidant parameters, total bilirubin, protein, lipid peroxidation, and liver cancer biomarkers.

An object of the present disclosure is to evaluate the chemo-preventive potential of PHC (2:1:1) and its prepared chitosan nanoparticles.

Another object of the present disclosure is to estimate the potential of selected polyherbal combination and its prepared chitosan nanoparticles against DEN induced liver tumors in rats.

Another object of the present disclosure is to deliver new perceptions into the development of different formulations of PHC with a ratio of 2:1:1 which can act as targeted preventive therapies for hepatocellular carcinoma.

Yet another object of the present invention is to deliver an expeditious and cost-effective system for treating cancer and evaluating chemo-preventive potential of PHC and its prepared chitosan nanoparticles.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a block diagram of a system for treating cancer and evaluating chemo-preventive potential of PHC and its prepared chitosan nanoparticles in accordance with an embodiment of the present disclosure;

FIG. 2 illustrates effect on Body weight in control and experimental groups in accordance with an embodiment of the present disclosure;

FIG. 3 illustrates effect on Liver weight and Relative liver weight in control and experimental groups in accordance with an embodiment of the present disclosure;

FIG. 4 illustrates effect on Enzymatic parameters (AST, ALT, ALP and LDH) in control and experimental groups in accordance with an embodiment of the present disclosure;

FIG. 5 illustrates effect on Antioxidant Parameters (SOD, CAT and GPx) in control and experimental groups in accordance with an embodiment of the present disclosure;

FIG. 6 illustrates effect on total bilirubin, total protein, albumin and globulin in control and experimental groups in accordance with an embodiment of the present disclosure;

FIG. 7 illustrates effect on lipid peroxidation (LPO) in control and experimental groups in accordance with an embodiment of the present disclosure;

FIG. 8 illustrates effect on Cancer biomarkers (AFP & CEA) in control and Experimental groups in accordance with an embodiment of the present disclosure;

FIG. 9 illustrates representative Western blot and expression of GST-P protein levels in accordance with an embodiment of the present disclosure;

FIG. 10 illustrates Table 1 depicts treatment protocol; and

FIG. 11 illustrates Table 2 depicts summary of tumour data showing effects of chitosan nanoparticles treatment on the DEN induced hepatocellular carcinoma in rats.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

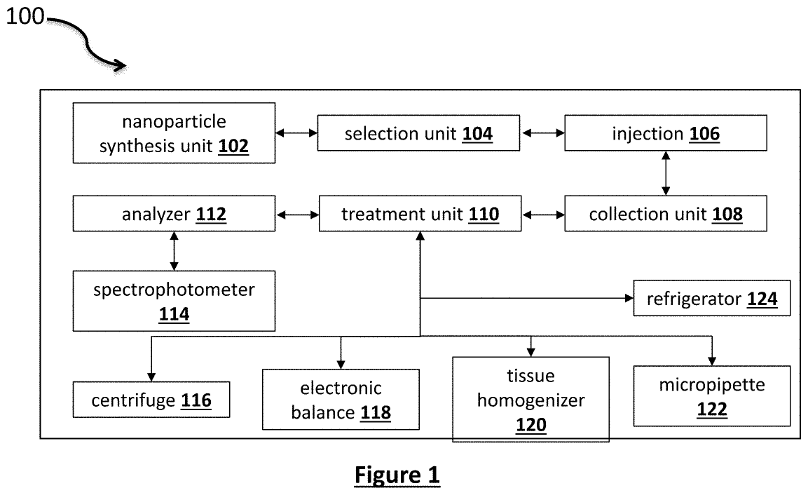

Referring to FIG. 1, a block diagram of a system for treating cancer and evaluating chemo-preventive potential of PHC and its prepared chitosan nanoparticles is illustrated in accordance with an embodiment of the present disclosure. The system 100 includes a nanoparticle synthesis unit 102 for synthesizing chitosan nanoparticles using polyherbal combination of ratio (2:1:1).

In an embodiment, a selection unit 104 is employed for selecting a group of rats each weighing 125-150 grams and accommodating in polypropylene cages under proper environmental conditions at room temperature of 23±2° C. and 50-60% relative humidity followed by 12 hours light/dark cycle.

In an embodiment, an injection 106 is used for inducing Hepatocellular Carcinoma (HCC), wherein for the induction of HCC, rats administered single intraperitoneal injection of DEN (200 mg/kg body weight) followed by weekly subcutaneous injections of carbon tetrachloride (3 ml/kg body weight) for 6 weeks, wherein the rats are divided into eight groups, from which group 1 is served as normal control, and group 2-8 are given single dose of DEN and repeated dose of CCl₄ till the completion of the experimental duration of 16 weeks, wherein freshly prepared solution of DEN in normal saline is used for the induction of HCC in rats by administering 200 mg/kg, i.p., PHC (2:1:1) is dispersed in normal saline suspension to administer at doses of 900 mg/kg, p.o and 1935 mg/kg p.o. Soranib (Sorafenib 200 mg) tablets are powdered and dissolve in normal saline solution and the freshly prepared solution is used as standard drug.

In an embodiment, a collection unit 108 is used for collecting serum and tissue samples after anesthetizing overnight fasted rats using intraperitoneal administration of thiopentone sodium at a dose of 40 mg/kg, wherein the blood sample is collected by retro-orbital plexus followed by cardiac puncture.

In an embodiment, a treatment unit 110 is used for treating the collected serum and tissue samples.

In an embodiment, an analyzer 112 is equipped with a spectrophotometer 114 for evaluating the chemo-preventive potential of PHC (2:1:1) and its prepared chitosan nanoparticles upon determining liver markers, antioxidant parameters, total bilirubin, protein, lipid peroxidation, and liver cancer biomarkers.

In another embodiment, all the rats are allowed to consume commercial pellet diet during the course of the study, wherein eight groups of rat are differentiated into six groups consisting of eight rats and two groups consisting of twelve rats.

In another embodiment, the polyherbal combination composition is selected from a group containing: Carbon tetrachloride (CCl₄), Disodium hydrogen ortho-phosphate, Distilled water, Ethylenediaminetetraacetic acid, Formaldehyde solution, Hydrogen peroxide, Thiopentone Sodium (Intraval Sodium injection), N-Nitrosodiethylamine (DEN), Normal Saline (0.9%), Potassium Dihydrogen orthophosphate, Pyrogallol, Sorafenib, and Tris Hydrochloride buffer.

In another embodiment, the treatment unit 110 comprises a centrifuge 116 for centrifugating the collected sample of blood at 3000 rpm for 10 minutes to separate the serum, which is kept at −20° C. until further analysis.

In one embodiment, an electronic balance 118 is coupled to the treatment unit 110 for weighing washed and blotted dried liver tissue, wherein the liver tissue is washed two times ice-cold normal saline.

In one embodiment, a tissue homogenizer 120 is coupled to the treatment unit 110 for preparing ten percentage homogenates of liver tissue.

In one embodiment, a micropipette 122 is coupled to the treatment unit 110 for pouring formalin solution for histopathological examination.

In one embodiment, a refrigerator 124 is coupled to the treatment unit 110 for storing the tissues at −20° C. and observing the presence of nodules using the UV spectrophotometer 114.

In another embodiment, the live markers are selected from a group comprising Alanine Aminotransaminase (ALT)/Serum Glutamate Pyruvate Transaminase (SGPT), Aspartate Aminotransaminase (AST)/Serum Glutamate Oxaloacetate Transaminase (SGOT), Alkaline Phosphatase (ALP), Lactate dehydrogenase (LDH), total protein, albumin, globulin and total bilirubin.

In another embodiment, various working reagent is prepared for determining the liver markers, wherein 1 ml of Serum and 4 ml of AST reagent is mixed to prepare the working reagent for Aspartate transaminase (AST/SGOT) and Alanine transaminase (ALT or SGPT), wherein 20 μl of Serum and 1000 μl of AST reagent is mixed to prepare the working reagent for Alkaline phosphatase (ALP), wherein working reagent is prepared by combining 0.8 ml of Buffer reagent and 0.2 ml of Starter reagent.

In another embodiment, the antioxidant parameters are selected from a group comprising superoxide dismutase (SOD) activity, Catalase (CAT) activity, and glutathione peroxidase (GPx).

In another embodiment, various working reagent is prepared for determining the antioxidant parameters, wherein 186 mg of EDTA and 788 mg of Tris HCl buffer is mixed in 100 mL doubled distilled water thereby in 5 mL of 10 mM mix, dissolve HCl 15.1 mg of pyrogallol preparation of the reagents for assessment of superoxide dismutase (SOD) activity, wherein 6.81 g of KH₂PO₄.2H₂O is dissolved in double distilled water and the final volume is made up to 1000 ml to prepare potassium di-hydrogen phosphate (KH₂PO₄.2H₂O), 8.9 g of Na₂HPO₄.2H₂O is dissolved in double distilled water and the final volume is made up to 1000 ml for preparing Disodium hydrogen phosphate (Na₂HPO₄.2H₂O), mixing KH₂PO₄.2H₂O and Na₂HPO₄.2H₂O in 1:1 ration for preparing Potassium phosphate buffer, and 187 μl of 30% H₂O₂ is dissolved in 100 ml phosphate buffer for preparing 30% Hydrogen peroxide solution for estimation of Catalase (CAT) activity, and wherein reagents 10 mM Sodium azide, 0.4 MTris buffer with a pH 7.0, 4-mM Reduced glutathione, 2.5 mM Hydrogen peroxide, 4 mM EDTA, 10% of TCA, Ellman's reagent: 0.04% DTNB dissolved in 1% sodium citrate, 0.3 M Phosphate solution (Dibasic (Na₂HPO₄) having conc. of 0.3M, pH 8.0), and reduced glutathione standard: In 100 ml of water dissolve 20 mg of reduced glutathione for estimation of glutathione peroxidase (GPx) (EC 1.11.1.9).

In another embodiment, the reagents for assessment of lipid peroxidation (liver tissues) is selected from a group of Thiobarbituric acid (TBA) solution (0.8%), Trichloroacetic acid (TCA) solution (30%), and Potassium chloride solution (1.15% w/v of KCl), wherein the 0.8 g of TBA is dissolved in distilled water and volume is made up to 100 ml for preparing Thiobarbituric acid (TBA) solution (0.8%), wherein 30 g of TCA is dissolved in distilled water and volume is made up to 100 ml for preparing Trichloroacetic acid (TCA) solution (30%).

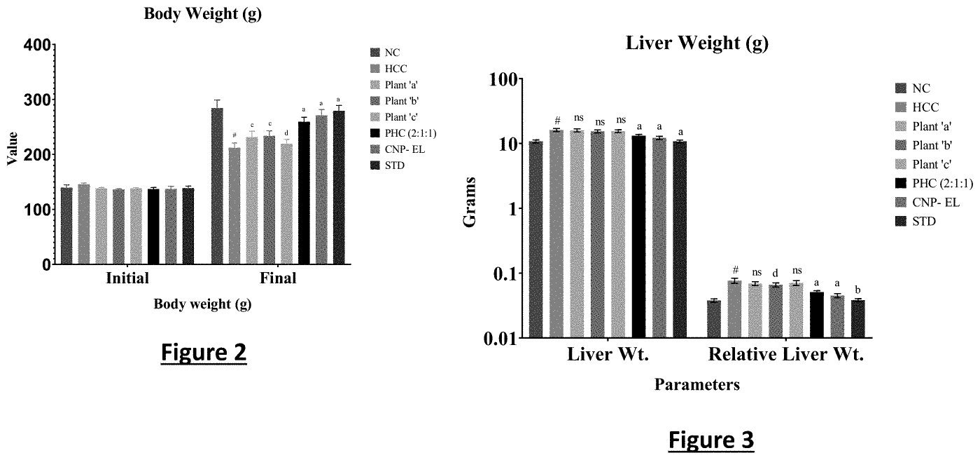

FIG. 2 illustrates effect on Body weight in control and experimental groups in accordance with an embodiment of the present disclosure. All values are expressed as mean±Standard deviation (SD) (n=6) except n=5 in Plant ‘c’ group. Statistical analysis is done using One way Analysis of Variance (ANOVA) followed by Dunnett's t-test using GraphPad Prism Version 8.0.1. Where, HCC is compared with NC group while the treatment groups (Plant ‘a’, Plant ‘b’, Plant ‘c’, PHC (2:1:1), CNP-EL and STD) are compared with HCC group. ^{#}P<0.0001 is very highly significant when compared with NC, ^{a}P<0.0001 is very highly significant, ^{c}P<0.01 is very significant and ^{d}P<0.05 significant when compared with HCC group. Where NC is normal control group; HCC is hepatocellular carcinoma group; Plant ‘a’ group is Berberis aristata treated group; Plant ‘b’ group is Andrographis paniculata treated group; Plant ‘c’ group is Thevetia peruviana treated group; PHC (2:1:1) group is Polyherbal combination of 2:1:1 treated group; CNP-EL group is Chitosan nanoparticles of PHC (2:1:1) treated group and STD group is Standard Sorafenib treated group.

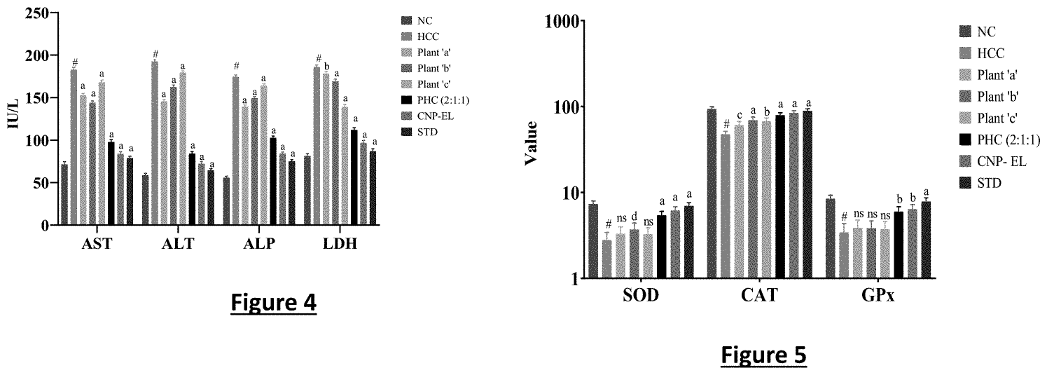

FIG. 3 illustrates effect on Liver weight and Relative liver weight in control and experimental groups in accordance with an embodiment of the present disclosure. All values are expressed as mean±Standard deviation (SD) (n=6) except n=5 in Plant ‘c’ group. Statistical analysis is done using One way Analysis of Variance (ANOVA) followed by Dunnett's t-test using GraphPad Prism Version 8.0.1. Where, HCC is compared with NC group while the treatment groups (Plant ‘a’, Plant ‘b’, Plant ‘c’, PHC (2:1:1), CNP-EL and STD) are compared with HCC group. ^{#}P<0.0001 is very highly significant when compared with NC, ^{a}P<0.0001 is very highly significant, ^{b}P<0.001 is highly significant, ^{d}P<0.05 is significant and ns=not significant when compared with HCC group. Where NC is normal control group; HCC is hepatocellular carcinoma group; Plant ‘a’ group is Berberis aristata treated group; Plant ‘b’ group is Andrographis paniculata treated group; Plant ‘c’ group is Thevetia peruviana treated group; PHC (2:1:1) group is Polyherbal combination of 2:1:1 treated group; CNP-EL group is Chitosan nanoparticles of PHC (2:1:1) treated group and STD group is Standard Sorafenib treated group.

In an embodiment, all the eight groups of Wistar rats showing the nodules incidence and animal survival that are presented in Table 2 and FIG. 4. Macroscopically visible hepatic nodules in rats are shown in FIG. 4 that represent livers are removed out from several groups respectively. In normal control group (NC) (Group 1) showed absence of any kind of nodules where as in hepatocellular carcinoma control group (HCC) (Group 2) showed 6-7 number nodules. Treatment with Plant ‘a’ (Group 3) and PHC (2:1:1) (Group 6) showed 3-4 number of small nodules while treatment with ‘b’ (Group 4) and ‘c’ (Group 5) showed 5-6 number of nodules. Treatment with CNP-EL (Group 7) showed 2-3 number of nodules while STD group (Group 8) showed 0-1 number of nodules. Orange arrow shows the presence of macroscopic nodules.

FIG. 4 illustrates effect on Enzymatic parameters (AST, ALT, ALP and LDH) in control and experimental groups in accordance with an embodiment of the present disclosure. All values are expressed as mean±Standard deviation (SD) (n=6) except n=5 in Plant ‘c’ group. Statistical analysis is done using One way Analysis of Variance (ANOVA) followed by Dunnett's t-test using GraphPad Prism Version 8.0.1. Where, HCC is compared with NC group while the treatment groups (Plant ‘a’, Plant ‘b’, Plant ‘c’, PHC (2:1:1), CNP-EL and STD) are compared with HCC group. ^{#}P<0.0001 is very highly significant when compared with NC, ^{a}P<0.0001 is very highly significant, ^{b}P<0.001 is highly significant when compared with HCC group. Where NC is normal control group; HCC is hepatocellular carcinoma group; Plant ‘a’ group is Berberis aristata treated group; Plant ‘b’ group is Andrographis paniculata treated group; Plant ‘c’ group is Thevetia peruviana treated group; PHC (2:1:1) group is Polyherbal combination of 2:1:1 treated group; CNP-EL group is Chitosan nanoparticles of PHC (2:1:1) treated group and STD group is Standard Sorafenib treated group. Units: AST, ALT, ALP and LDH (IU/L).

FIG. 5 illustrates effect on Antioxidant Parameters (SOD, CAT and GPx) in control and experimental groups in accordance with an embodiment of the present disclosure. All values are expressed as mean±Standard deviation (SD) (n=6) except n=5 in Plant ‘c’ group. Statistical analysis is done using One way Analysis of Variance (ANOVA) followed by Dunnett's t-test using GraphPad Prism Version 8.0.1. Where, HCC is compared with NC group while the treatment groups (Plant ‘a’, Plant ‘b’, Plant ‘c’, PHC (2:1:1), CNP-EL and STD) are compared with HCC group. ^{#}P<0.0001 is very highly significant when compared with NC, ^{a}P<0.0001 is very highly significant, ^{b}P<0.001 is highly significant, ^{c}P<0.01 is very significant, ^{d}P<0.05 is significant and ns=not significant when compared with HCC group. Where NC is normal control group; HCC is hepatocellular carcinoma group; Plant ‘a’ group is Berberis aristata treated group; Plant ‘b’ group is Andrographis paniculata treated group; Plant ‘c’ group is Thevetia peruviana treated group; PHC (2:1:1) group is Polyherbal combination of 2:1:1 treated group; CNP-EL group is Chitosan nanoparticles of PHC (2:1:1) treated group and STD group is Standard Sorafenib treated group. Units: SOD: Units/mg of protein; CAT: nmoles of H₂O₂ consumed/min/mg of protein and μmol GSH Oxidized/min/mg of protein.

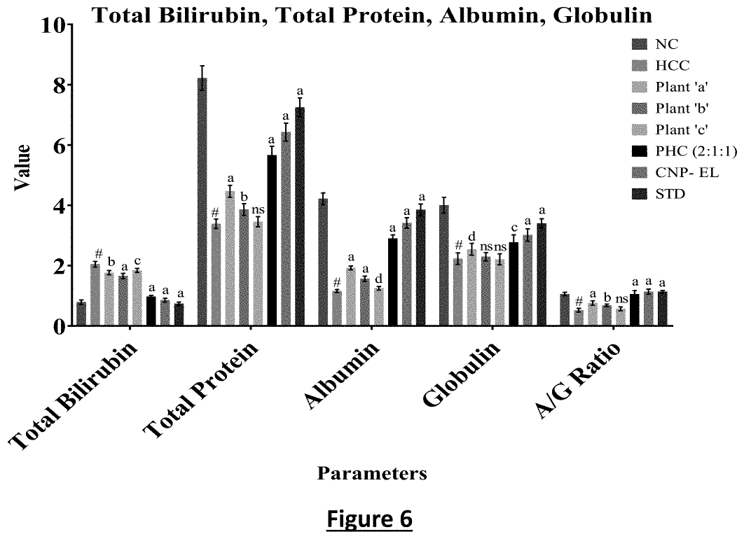

FIG. 6 illustrates effect on total bilirubin, total protein, albumin and globulin in control and experimental groups in accordance with an embodiment of the present disclosure. All values are expressed as mean±Standard deviation (SD) (n=6) except n=5 in Plant ‘c’ group. Statistical analysis is done using One way Analysis of Variance (ANOVA) followed by Dunnett's t-test using GraphPad Prism Version 8.0.1. Where, HCC is compared with NC group while the treatment groups (Plant ‘a’, Plant ‘b’, Plant ‘c’, PHC (2:1:1), CNP-EL and STD) are compared with HCC group. ^{#}P<0.0001 is very highly significant when compared with NC, ^{a}P<0.0001 is very highly significant, ^{b}P<0.001 is highly significant, ^{c}P<0.01 is very significant, ^{d}P<0.05 is significant and ns=not significant when compared with HCC group. Where NC is normal control group; HCC is hepatocellular carcinoma group; Plant ‘a’ group is Berberis aristata treated group; Plant ‘b’ group is Andrographis paniculata treated group; Plant ‘c’ group is Thevetia peruviana treated group; PHC (2:1:1) group is Polyherbal combination of 2:1:1 treated group; CNP-EL group is Chitosan nanoparticles of PHC (2:1:1) treated group and STD group is Standard Sorafenib treated group. Unit: Bilirubin: mg/dl, Total protein, Albumin and globulin: gm/dl.

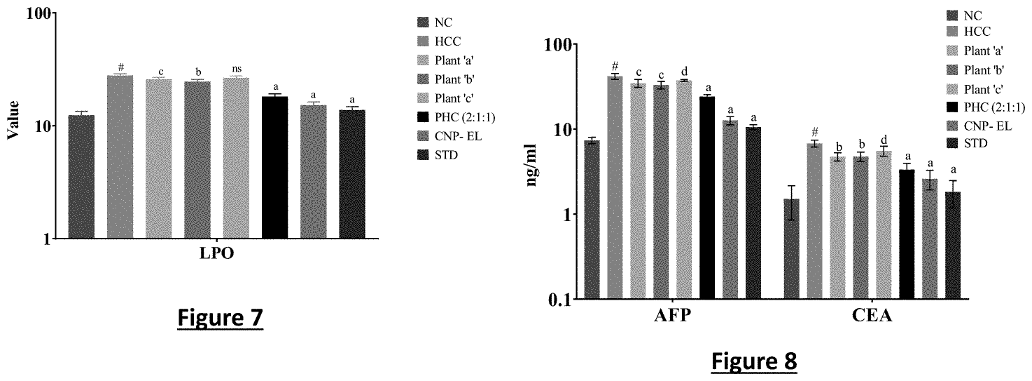

FIG. 7 illustrates effect on lipid peroxidation (LPO) in control and experimental groups in accordance with an embodiment of the present disclosure. All values are expressed as mean±Standard deviation (SD) (n=6) except n=5 in Plant ‘c’ group. Statistical analysis is done using One way Analysis of Variance (ANOVA) followed by Dunnett's t-test using GraphPad Prism Version 8.0.1. Where, HCC is compared with NC group while the treatment groups (Plant ‘a’, Plant ‘b’, Plant ‘c’, PHC (2:1:1), CNP-EL and STD) are compared with HCC group. ^{#}P<0.0001 is very highly significant when compared with NC, ^{a}P<0.0001 is very highly significant, ^{b}P<0.001 is highly significant, ^{c}P<0.01 is very significant and ns=not significant when compared with HCC group. Where NC is normal control group; HCC is hepatocellular carcinoma group; Plant ‘a’ group is Berberis aristata treated group; Plant ‘b’ group is Andrographis paniculata treated group; Plant ‘c’ group is Thevetia peruviana treated group; PHC (2:1:1) group is Polyherbal combination of 2:1:1 treated group; CNP-EL group is Chitosan nanoparticles of PHC (2:1:1) treated group and STD group is Standard Sorafenib treated group. Unit: nmoles/min/mg of protein.

FIG. 8 illustrates effect on Cancer biomarkers (AFP & CEA) in control and Experimental groups in accordance with an embodiment of the present disclosure. All values are expressed as mean±Standard deviation (SD) (n=6) except n=5 in Plant ‘c’ group. Statistical analysis is done using One way Analysis of Variance (ANOVA) followed by Dunnett's t-test using GraphPad Prism Version 8.0.1. Where, HCC is compared with NC group while the treatment groups (Plant ‘a’, Plant ‘b’, Plant ‘c’, PHC (2:1:1), CNP-EL and STD) are compared with HCC group. ^{#}P<0.0001 is very highly significant when compared with NC, ^{a}P<0.0001 is very highly significant, ^{b}P<0.001 is highly significant, ^{c}P<0.01 is very significant and ^{d}P<0.05 is significant when compared with HCC group. Where NC is normal control group; HCC is hepatocellular carcinoma group; Plant ‘a’ group is Berberis aristata treated group; Plant ‘b’ group is Andrographis paniculata treated group; Plant ‘c’ group is Thevetia peruviana treated group; PHC (2:1:1) group is Polyherbal combination of 2:1:1 treated group; CNP-EL group is Chitosan nanoparticles of PHC (2:1:1) treated group and STD group is Standard Sorafenib treated group. Units: AFP=ng/ml; CEA=ng/ml.

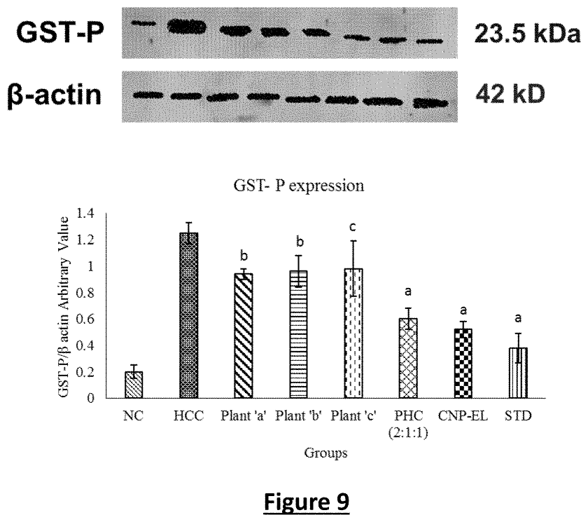

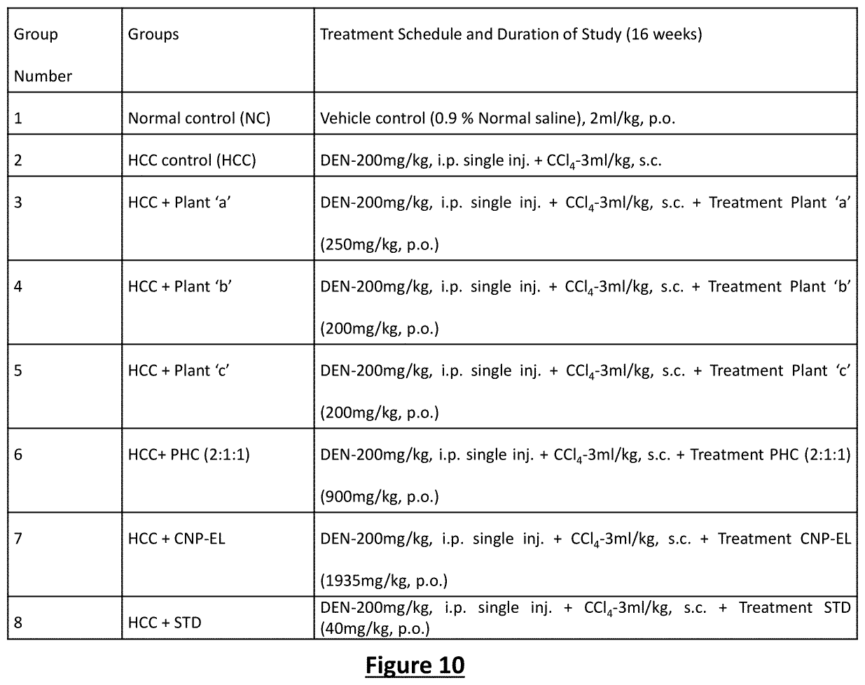

FIG. 9 illustrates representative Western blot and expression of GST-P protein levels in accordance with an embodiment of the present disclosure. All values are expressed as mean±Standard deviation (SD) (n=6) except n=5 in Plant ‘c’ group. Statistical analysis is done using One way Analysis of Variance (ANOVA) followed by Dunnett's t-test using GraphPad Prism Version 8.0.1. Where, HCC is compared with NC group while the treatment groups (Plant ‘a’, Plant ‘b’, Plant ‘c’, PHC (2:1:1), CNP-EL and STD) are compared with HCC group. ^{#}P<0.0001 is very highly significant when compared with NC, ^{a}P<0.0001 is very highly significant, ^{b}P<0.001 is highly significant and ^{c}P<0.01 is very significant when compared with HCC group. Where NC is normal control group; HCC is hepatocellular carcinoma group; Plant ‘a’ group is Berberis aristata treated group; Plant ‘b’ group is Andrographis paniculata treated group; Plant ‘c’ group is Thevetia peruviana treated group; PHC (2:1:1) group is Polyherbal combination of 2:1:1 treated group; CNP-EL group is Chitosan nanoparticles of PHC (2:1:1) treated group and STD group is Standard Sorafenib treated group.

In an embodiment, (NC) (40×) H and E stained section of liver from normal group (Group 1) rat showing normal hepatic cells with well-preserved/granular cytoplasm; brought out central vein; prominent nucleus and nucleolus. (HCC) (40×) H and E stained section of liver from HCC group (Group 2) rat showing irregular cytoplasm shape, cell necrosis, loss of lobular architecture, loss of parenchymal portal triad. (Plant ‘a’) (40×) H and E stained section of liver from Plant ‘a’ treated group (Group 3) rat showing mild distorted architecture, less cellular swelling with mild inflammatory cell infiltration. (Plant ‘b’) (40×) H and E stained section of liver from Plant ‘b’ treated group (Group 4) rat showing moderate distortion of the hepatic architecture like presence of minimum inflammatory changes around the portal triads. (Plant ‘c’) (40×) H and E stained section of liver from Plant ‘c’ treated group (Group 5) rat showing mild disruption in the architecture with absence of proliferation of hepatocytes. (PHC 2:1:1) (40×) H and E stained section of liver from PHC (2:1:1) treated group (Group 6) rat showing normal hepatic cells with lymphocytic without any disturbances of hepatic architecture. (CNP-EL) (40×) H and E stained section of liver from CNP-EL treated group (Group 7) rat showing normal cytoplasm shape, reduced number of binucleated cells and hepatic tissue restoration with altered hepatocytes. (STD) (40×) H and E stained section of liver from STD treated group (Group 8) rat showing hepatic tissue with cytoplasm, less necrosis of cell, altered hepatocytes and hepatocellular architecture. Where, Plant ‘a’ is Berberis aristata; Plant ‘b’ is Andrographis paniculata; Plant ‘c’ is Thevetia peruviana; PHC (2:1:1) is Polyherbal combination of 2:1:1; CNP-EL is Chitosan nanoparticles of PHC (2:1:1) and STD is Standard Sorafenib.

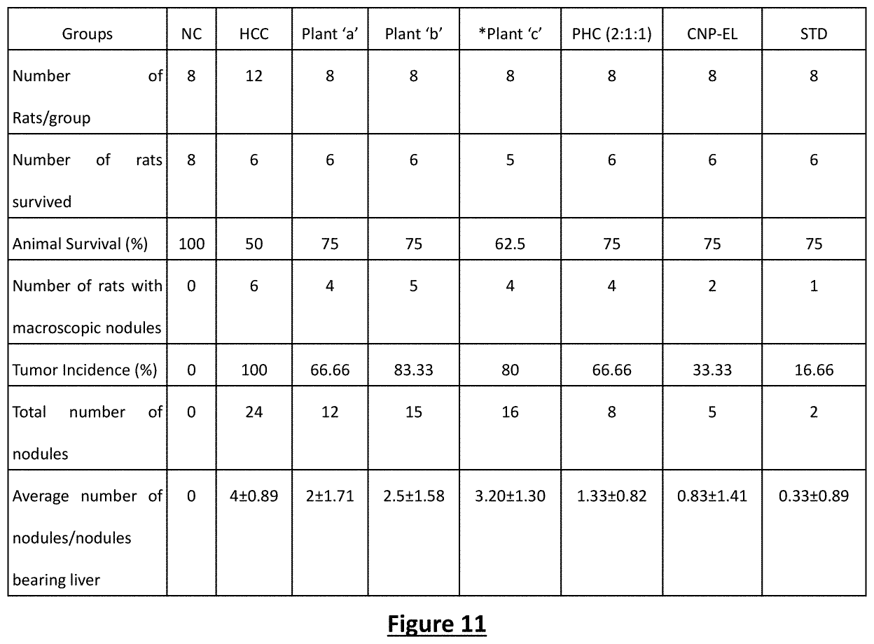

FIG. 10 illustrates Table 1 depicts treatment protocol.

FIG. 11 illustrates Table 2 depicts summary of tumour data showing effects of chitosan nanoparticles treatment on the DEN induced hepatocellular carcinoma in rats.

In FIG. 7 the levels of total bilirubin, total protein, albumin, globulin and A/G ratio are observed to be statistically very highly significant (^{#}P<0.0001) in HCC group (Group 2) when compared to normal control group (Group 1). In Plant ‘a’ treated group (Group 3), the level of total bilirubin is statistically highly significant (^{b}P<0.001) decreased when compared to HCC group (Group 2) but the levels of total protein, albumin and A/G ratio are found to be statistically very highly significant (^{a}P<0.0001) increased while, the levels of globulin are found to be statistically significant (^{d}P<0.05) increased when compared to the HCC group (Group 2). In Plant ‘b’ treated group (Group 4), the levels of total bilirubin is found to be statistically very highly significant (^{a}P<0.0001) decreased while, the levels of total protein, albumin, globulin and A/G ratio are found to be statistically highly significant (^{b}P<0.001), very highly significant (^{a}P<0.0001), not significant (^{ns}P>0.05) and highly significant (P<0.001) respectively when compared to HCC group (Group 2). Further, in Plant ‘c’ treated group (Group 5), there is decreased in the level of total bilirubin which is found to be statistically very significant (^{c}P<0.01) whereas, total protein, globulin and A/G ratio are found to be statistically not significant (^{ns}P>0.05) when compared to HCC group (Group 2) whereas, a statistically significant (^{d}P<0.05) increase in the levels of albumin are observed when compared to HCC group (Group 2). Moreover, in PHC (2:1:1) treated group (Group 6), the levels of total bilirubin is found to be statistically very highly significantly (^{a}P<0.0001) decreased whereas the levels of total protein, albumin and A/G ratio are found to be statistically very highly significant (^{a}P<0.0001) increased when compared to HCC control group (Group 2) while, there is statistically very significant (^{c}P<0.01) increased in the levels of globulin when compared to HCC group (Group 2). Additionally, in CNP-EL and STD treated groups (Group 7 and 8), the level of total bilirubin is found to be statistically very highly significantly (^{a}P<0.0001) decreased whereas, the levels of total protein, albumin, globulin and A/G ratio are found to be statistically very highly significant (^{a}P<0.0001) increased when compared to HCC group (Group 2) as shown in FIG. 7.

NDEA also showed that, on metabolic biotransformation, it produces premutagenic products, O6-ethyldeoxyguanosine and O4 and O6-ethyldeoxythymidine in liver that are responsible for their carcinogenic effects. It is well documented that CCl₄ encourages hepatotoxicity by metabolic activation and hence selectively leads to toxicity in hepatocytes sustaining a semi-normal metabolic function. In the endoplasmic reticulum, CCl₄ is bio-transformed through cytochrome P450 (CYP) enzyme system to produce trichloromethyl free radicals. After that, trichloromethyl free radicals interfere with cellular lipids and proteins in the occurrence of oxygen to generate a trichloromethyl peroxyl radical that may further attack lipids on the endoplasmic reticulum membrane quicker than trichloromethyl free radical. Consequently, trichloromethyl peroxyl free radical resulted in the production of lipid peroxidation (LPO) and devastation of Ca²⁺ homeostasis that results in cell death. The level of lipid peroxidation (LPO) can indicate the status of oxidative damage in the liver tissues. Lipid peroxidation as well as a main variety of harmful products have been recommended as a chief source of cancer development. Administration of N-Nitrosodiethylamine (NDEA) has been described to produce lipid peroxidation products and activated oxygen species during hepatocarcinogenesis.

Malondialdehyde (MDA), an end product of LPO is assessed in the tumor tissues homogenates. The levels of MDA are observed to be statistically very highly significant (^{#}P<0.0001) increased in HCC group (Group 2) when compared to normal control group (Group 1) as represented in FIG. 8 Some researchers reported that increased lipid peroxidation and declined antioxidant defenses in the liver of a rat model of DEN induced hepatocarcinogenesis which is similar to our findings. However, in Plant ‘a’ and Plant ‘b’ treated group (Group 3 & 4) there is very significant (^{c}P<0.01), highly significant (^{b}P<0.001) decreased in the levels of MDA while in Plant ‘c’ treated group (Group 5) showed no significant (^{ns}P>0.05) differences in the levels of MDA when compared to HCC group (Group 2). In PHC (2:1:1), CNP-EL and STD treated groups (Group 6, 7 & 8) levels of MDA are very highly significant (^{a}P<0.0001) decreased when compared to HCC group (Group 2).

The most extensively used tumor marker for detection/diagnosis of HCC is alpha feto protein (AFP), which is a distinctive immunomodulatory glycoprotein (65 kDa) typically prepared by the immature hepatic cells in the fetus. It is a specific tumor indicator frequently used for the initial detection of hepatic cancer and has been significantly augmented in both early as well as advanced DEN induced HCC. It has been documented that exposure of rats to a certain carcinogens like NDEA/DEN increases the circulating levels of AFP. This validates the results that showed noteworthy elevation in the levels of AFP achieved in NDEA-induced rats that are noted to be reduced in treated groups (Groups 3 to 8). AFP too connects closely with the progression/growth rate (number of dividing cells) as well as size of the tumor along with progressive advancement of alpha fetoprotein in biopsied liver samples of patients with hepatic cirrhosis and hepatocellular carcinoma. In patients, its re-expression with HCC recommends abnormal or transformed liver cell regeneration, or differentiation of hepatic cells into tumor cells.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A system for evaluating chemo-preventive potential of PHC and its prepared chitosan nanoparticles, the system comprises:
a nanoparticle synthesis unit for synthesizing chitosan nanoparticles using polyherbal combination of ratio (2:1:1);
a selection unit for selecting a group of rats each weighing 125-150 grams and accommodating in polypropylene cages under proper environmental conditions at room temperature of 23±2° C. and 50-60% relative humidity followed by 12 hours light/dark cycle;
an injection for inducing Hepatocellular Carcinoma (HCC), wherein for the induction of HCC, rats administered single intraperitoneal injection of DEN (200 mg/kg body weight) followed by weekly subcutaneous injections of carbon tetrachloride (3 ml/kg body weight) for 6 weeks;
wherein the rats are divided into eight groups, from which group 1 is served as normal control, and group 2-8 are given single dose of DEN and repeated dose of CCl₄ till the completion of the experimental duration of 16 weeks;
wherein freshly prepared solution of DEN in normal saline is used for the induction of HCC in rats by administering 200 mg/kg, i.p., PHC (2:1:1) is dispersed in normal saline suspension to administer at doses of 900 mg/kg, p.o and 1935 mg/kg p.o. Soranib (Sorafenib 200 mg) tablets are powdered and dissolve in normal saline solution and the freshly prepared solution is used as standard drug;
a collection unit for collecting serum and tissue samples after anesthetizing overnight fasted rats using intraperitoneal administration of thiopentone sodium at a dose of 40 mg/kg, wherein the blood sample is collected by retro-orbital plexus followed by cardiac puncture;
a treatment unit for treating the collected serum and tissue samples; and
an analyzer equipped with a spectrophotometer for evaluating the chemo-preventive potential of PHC (2:1:1) and its prepared chitosan nanoparticles upon determining liver markers, antioxidant parameters, total bilirubin, protein, lipid peroxidation, and liver cancer biomarkers.

2. The system of claim 1, wherein all the rats are allowed to consume commercial pellet diet during the course of the study, wherein eight groups of rat are differentiated into six groups consisting of eight rats and two groups consisting of twelve rats.

3. The system of claim 1, wherein the polyherbal combination composition is selected from a group containing: Carbon tetrachloride (CCl₄), Disodium hydrogen ortho-phosphate, Distilled water, Ethylenediaminetetraacetic acid, Formaldehyde solution, Hydrogen peroxide, Thiopentone Sodium (Intraval Sodium injection), N-Nitrosodiethylamine (DEN), Normal Saline (0.9%), Potassium Dihydrogen orthophosphate, Pyrogallol, Sorafenib, and Tris Hydrochloride buffer.

4. The system of claim 1, wherein the treatment unit comprises:
a centrifuge for centrifugating the collected sample of blood at 3000 rpm for 10 minutes to separate the serum, which is kept at −20° C. until further analysis;
an electronic balance for weighing washed and blotted dried liver tissue, wherein the liver tissue is washed two times ice-cold normal saline;
a tissue homogenizer for preparing ten percentage homogenates of liver tissue;
a micropipette for pouring formalin solution for histopathological examination; and
a refrigerator for storing the tissues at −20° C. and observing the presence of nodules using the UV spectrophotometer.

5. The system of claim 1, wherein the live markers are selected from a group comprising Alanine Aminotransaminase (ALT)/Serum Glutamate Pyruvate Transaminase (SGPT), Aspartate Aminotransaminase (AST)/Serum Glutamate Oxaloacetate Transaminase (SGOT), Alkaline Phosphatase (ALP), Lactate dehydrogenase (LDH), total protein, albumin, globulin and total bilirubin.

6. The system of claim 5, wherein various working reagent is prepared for determining the liver markers, wherein 1 ml of Serum and 4 ml of AST reagent is mixed to prepare the working reagent for Aspartate transaminase (AST/SGOT) and Alanine transaminase (ALT or SGPT), wherein 20 μl of Serum and 1000 μl of AST reagent is mixed to prepare the working reagent for Alkaline phosphatase (ALP), wherein working reagent is prepared by combining 0.8 ml of Buffer reagent and 0.2 ml of Starter reagent.

7. The system of claim 1, wherein the antioxidant parameters are selected from a group comprising superoxide dismutase (SOD) activity, Catalase (CAT) activity, and glutathione peroxidase (GPx).

8. The system of claim 7, wherein working reagent is prepared for determining the antioxidant parameters, wherein 186 mg of EDTA and 788 mg of Tris HCl buffer is mixed in 100 mL doubled distilled water thereby in 5 mL of 10 mM mix, dissolve HCl 15.1 mg of pyrogallol preparation of the reagents for assessment of superoxide dismutase (SOD) activity, wherein 6.81 g of KH₂PO₄.2H₂O is dissolved in double distilled water and the final volume is made up to 1000 ml to prepare potassium di-hydrogen phosphate (KH₂PO₄.2H₂O), 8.9 g of Na₂HPO₄.2H₂O is dissolved in double distilled water and the final volume is made up to 1000 ml for preparing Disodium hydrogen phosphate (Na₂HPO₄.2H₂O), mixing KH₂PO₄.2H₂O and Na₂HPO₄.2H₂O in 1:1 ration for preparing Potassium phosphate buffer, and 187 μl of 30% H₂O₂ is dissolved in 100 ml phosphate buffer for preparing 30% Hydrogen peroxide solution for estimation of Catalase (CAT) activity, and wherein reagents 10 mM Sodium azide, 0.4 MTris buffer with a pH 7.0, 4-mM Reduced glutathione, 2.5 mM Hydrogen peroxide, 4 mM EDTA, 10% of TCA, Ellman's reagent: 0.04% DTNB dissolved in 1% sodium citrate, 0.3 M Phosphate solution (Dibasic (Na₂HPO₄) having conc. of 0.3M, pH 8.0), and reduced glutathione standard: In 100 ml of water dissolve 20 mg of reduced glutathione for estimation of glutathione peroxidase (GPx) (EC 1.11.1.9).

9. The system of claim 1, wherein the reagents for assessment of lipid peroxidation (liver tissues) is selected from a group of Thiobarbituric acid (TBA) solution (0.8%), Trichloroacetic acid (TCA) solution (30%), and Potassium chloride solution (1.15% w/v of KCl), wherein the 0.8 g of TBA is dissolved in distilled water and volume is made up to 100 ml for preparing Thiobarbituric acid (TBA) solution (0.8%), wherein 30 g of TCA is dissolved in distilled water and volume is made up to 100 ml for preparing Trichloroacetic acid (TCA) solution (30%).
